(19) United States
(12) Patent Application Publication
(10) Pub. No.: US 2015/0320340 A1
(43) Pub. Date: Nov. 12, 2015

(54) Method and System For Preventing Virus Related Diseases

(71) Applicant: Rajesh Verma, Jacksonville, FL (US)

(72) Inventor: Rajesh Verma, Jacksonville, FL (US)

(21) Appl. No.: 14/628,201

(22) Filed: Feb. 20, 2015

Related U.S. Application Data
(60) Provisional application No. 61/942,537, filed on Feb. 20, 2014.

(51) Int. Cl. A61B 5/11; A61B 5/00

(52) U.S. Cl. CPC A61B 5/1114; A61B 5/681; A61B 5/746; A61B 5/7455; A61B 5/74

(57) ABSTRACT

A method for preventing a viral related infection includes obtaining a sample from a person, assaying the sample to determine whether the person has been previously infected with a virus, and if the person has not been previously infected, providing the person with at least one sensor positioned to detect when a person's hand approaches a predetermined position related to the ground or their own body. By warning the person of undesired hand-to- face contacts, the person is able to reduce the incidence of viral related infections.

### CROSS-REFERENCE TO RELATED APPLICATIONS

This application claims the benefit of provisional patent application Ser. No. 61/942,537 filed Feb. 20, 2014 by the present inventor.

### FEDERALLY SPONSORED RESEARCH

Not Applicable

### SEQUENCE LISTING OR PROGRAM Not Applicable BACKGROUND

1. Field

This application relates to viral infection prevention, specifically by preventing contaminated surfaces from contacting the face.

2. Background-Prior Art

On any given day people accumulate germs on their hands from a variety of sources. This can include many sources of germs such as direct contact with other people, contaminated surfaces such as tables, escalator handholds, foods, and even animals such as the family dog or cat. Subsequent to these contacts, if people don't wash their hands frequently and use the correct technique, they can easily infect themselves by touching their eyes, nose, mouth, or food. Further, failure to wash their hands will render a person a carrier who spreads germs to others by touching those people directly or by touching surfaces which others contact, such as doorknobs, faucets, counters, etc. As a consequence of inadequate hand hygiene, especially in children, infectious diseases are commonly spread from one person to another. Everything from the common cold and flu to gastrointestinal disorders, such as infectious diarrhea, are easily communicated from one person to the next.

Influenza (the flu) is a contagious disease that is caused by 3 viruses, influenza A, B and C. It attacks the respiratory tract (nose, throat, and lungs). The flu is different from a cold. While both are caused by viruses, high fever, headaches and extreme exhaustion are much more common with the flu. The flu can also cause serious complications such as bronchitis and pneumonia for certain high-risk groups. Influenza outbreaks occur in each hemisphere of the globe at least once a year and are responsible for hundreds of thousands of deaths around the world every year. Currently, between three and five million cases of severe illness and up to 500,000 deaths worldwide are attributable to the flu. Tens of millions of people died from flu epidemics in the 20′h century. New strains of flu virus appear almost every year or so. Approximately 36,000 deaths and more than 200,000 hospitalizations are directly associated with influenza every year in the United States. If a strain with similar virulence to the 1918 flu epidemic emerged today, experts predict that it could kill between 50 and 80 million people. In April 2009 a novel flu strain evolved that combined genes from human, pig, and bird flu. On Jun. 11, 2009, the World Health Organization officially declared the outbreak to be a pandemic. Every year in the US, 5% to 20% of the population gets the flu, and over 200,000 are hospitalized.

Inadequate hand hygiene and improper hand washing techniques also contribute to food-related illnesses, such as salmonella and E. coli infection. According to the Centers for Disease Control and Prevention (CDC), as many as 76 million Americans get a food-borne illness each year. Of these, about 5,000 die as a result of their illness. Others experience the annoying signs and symptoms of nausea, vomiting and diarrhea. This chilling statistic could easily be reduced if food handlers and people eating prepared food, washed their hands using proper techniques and for sufficient time periods.

The common cold is in general initiated by viral infections by the so-called cold viruses, such as rhino virus, corona virus, coxsackie virus, RS-virus, echovirus or other cold viruses. On average, all human beings suffer 2 to 3 times a year from infections in the upper respiratory passages. The majority of common colds in the Fall are caused by rhinovirus infection, whereas the majority of common cold occurring in January, February and March are caused by Coronavirus infections. (The converse is true in the Southern hemisphere.) Allergic syndromes, for example asthma, may be initiated by common cold viruses, especially the rhinovirus. Up to 70-75% of all patients suffering from common colds have rhinovirus infections ongoing either as a single infection or co-infection. In humans, adenoviruses infections are common and cause acute upper respiratory tract infections, enteritis or conjunctivitis, as well as other diseases.

The average pre-school child experiences 6-10 upper respiratory infections or common colds per year whereas the average adult experiences 2-4. The effects of the common cold can be uncommonly disruptive, forcing otherwise normal persons to stay away from work, school, etc. Individuals who are at increased risks, such as individuals suffering from bronchitis or asthma, may also experience a life-threatening exacerbation of their underlying conditions. The average annual expenditure for various cold treatments exceeds USD $2 billion in the United States, with similar amounts being expended each year in Europe.

Most colds are viral and are the leading cause of visits to the doctor. Antibiotics are prescribed for more than 60 percent of common colds, despite bacteria being involved in less than a few percent of the cases. The over prescription of unwarranted antibiotics has led to antibiotic-resistant bacteria so that when truly needed to treat a bacterial infection, antibiotics may not be able to treat it. Some contend that antibiotics actually make colds worse by killing ‘friendly’ bacteria and creating an environment more hospitable to viral infections. Antibiotics can also have side effects such as diarrhea and yeast infections. Despite repeated contentions, herbal remedies, such as Echinacea, for colds and the flu have yet to be supported with scientific studies.

Individuals infected with the flu virus are potentially contagious for the length of time one has symptoms, up to 7-10 days following the beginning of illness, and the initial incubation period is 24-48 hours. Influenza is spread by coming into contact with mucus membranes. The flu may be spread when a person touches a surface that has flu viruses on it, such as a doorknob, and then touches their nose or mouth. A single cold virus can have 16 million offspring within 24 hours.

In the past, individuals were taught to “cover their mouths” when they sneezed or coughed, resulting in viruses being transferred to one's hands where they can survive for a significant amount of time. Thus, this social practice of a courtesy actually promulgated the spread of disease, rather than avoiding its spread. According to the Mayo Clinic, the transfer of germs from hands to eyes, nose and mouth is the primary source for getting sick. Wearing face masks has been tried in order to limit transmission of colds and flu viruses. With the exception of certain Asian cultures, however, this practice has not been well received. Respirators, which are tight-fitting masks that filter airborne particles, are also beneficial, but they can be uncomfortable to wear for long periods of time and are expensive and cumbersome.

Rhinovirus infections in normal persons are initiated by selected events, which can be considered to occur sequentially. The steps in the rhinovirus pathogenesis are believed to include viral entry into the outer nose, mucociliary transport of virus to the posterior pharynx, and initiation of infection in ciliated and non-ciliated epithelial cells of the upper airway. Viral replication peaks on average within 48 hours of initiation of infection and persists for up to 3 weeks; Infection is followed by activation of several inflammatory mechanisms, which may include release or induction of interleukins, bradykinins, prostaglandins and possibly histamine, including stimulation of parasympathetic reflexes. The resultant clinical illness includes rhino sinusitis, pharyngitis, and bronchitis, which on average lasts one week. A secondary bacterial or microbial infection may follow subsequently to the viral infection and a sustained and more serious inflammation may result. Air-way infections or allergic rhinitis and/or asthma may pose serious health problems as it can be potentially life-threatening for susceptible groups such as elderly people with chronic airway problems or persons suffering from a deficient immunity, such as AIDS-patients, cancer patients etc.

In view of the long felt but unsolved needs related to the above description of viral and bacterially related health concerns, there is a desperate need for a simple and effective system and method of avoiding the undesired infections, occurrences, and symptoms/syndromes associated with hand-to-face transmissions of disease. In particular, a long felt but unsolved need relates to preventing infection with viral agents that cause obesity and/or cancer.

There have been attempts to warn one of his/her hand approaching the face. U.S. Pat. No. 0,144,453 to Kovarik et al. (2011) discusses sensors placed in various places such as hats, glasses, necklaces, shirts, helmets, and chairs. However, this had some disadvantages:
(a) In many of the embodiments, a sensor is placed in various parts of the body and a signaling unit is placed elsewhere. This would require two devices to keep track of and would be bulky.
(b) The sensors used were not very discrete, as in the hat, helmet or glasses.
(c) The proximity sensor would give a false positive if another object came close to the sensor.
(d) In some embodiments, the sensor was placed in a fixed location, such as a chair. If a person was not in that specific location, the sensor would not warn the person.

### SUMMARY

The present invention addresses the problem described above. A device is worn on the wrist, hand, or arm. The device will alert an individual when the hand is in a position that might lead to touching the face.

### DRAWINGS

### Figures

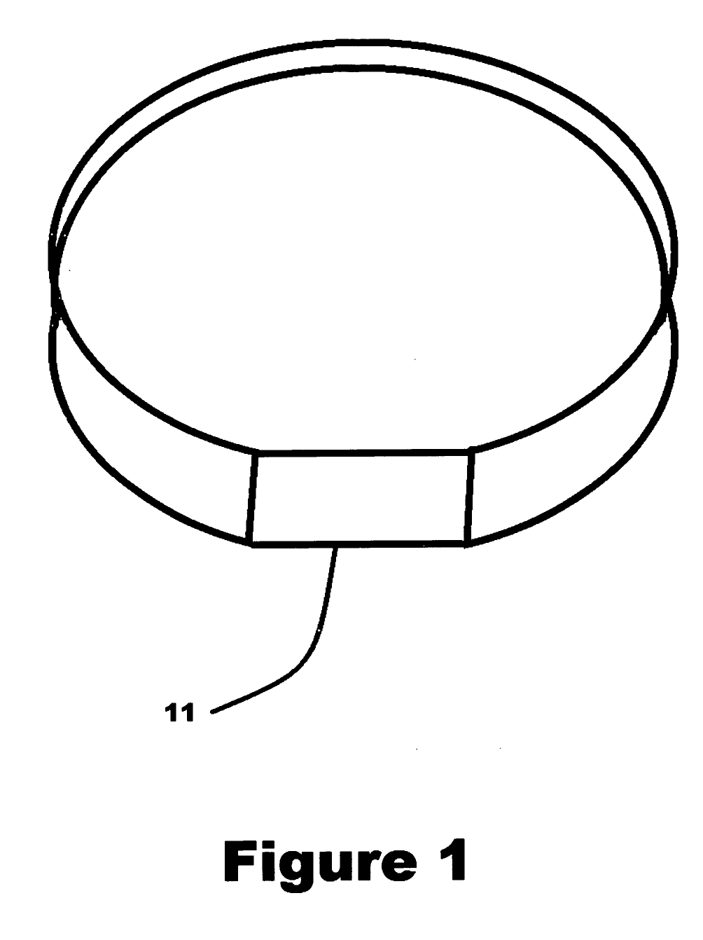

FIG. 1 shows a device that that finds the position of the hand.

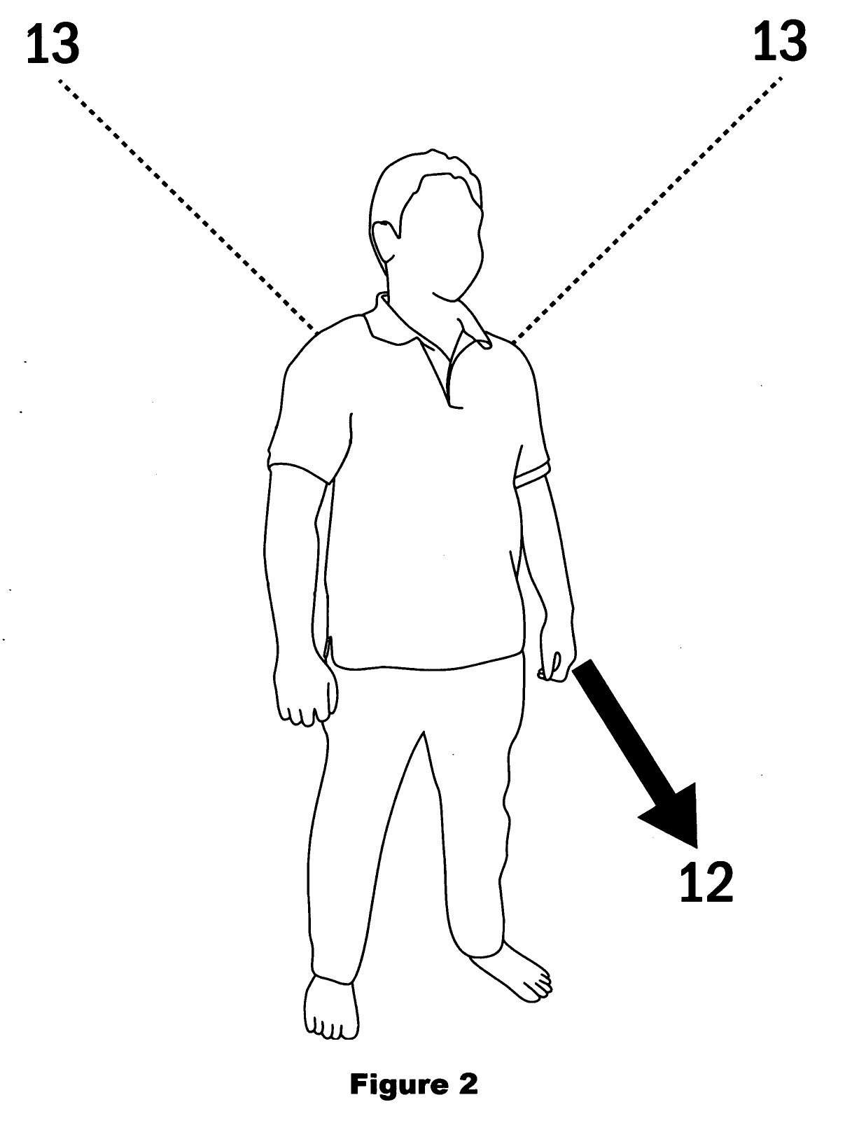

FIG. 2 shows a person standing and the relative position of the hand to the face.

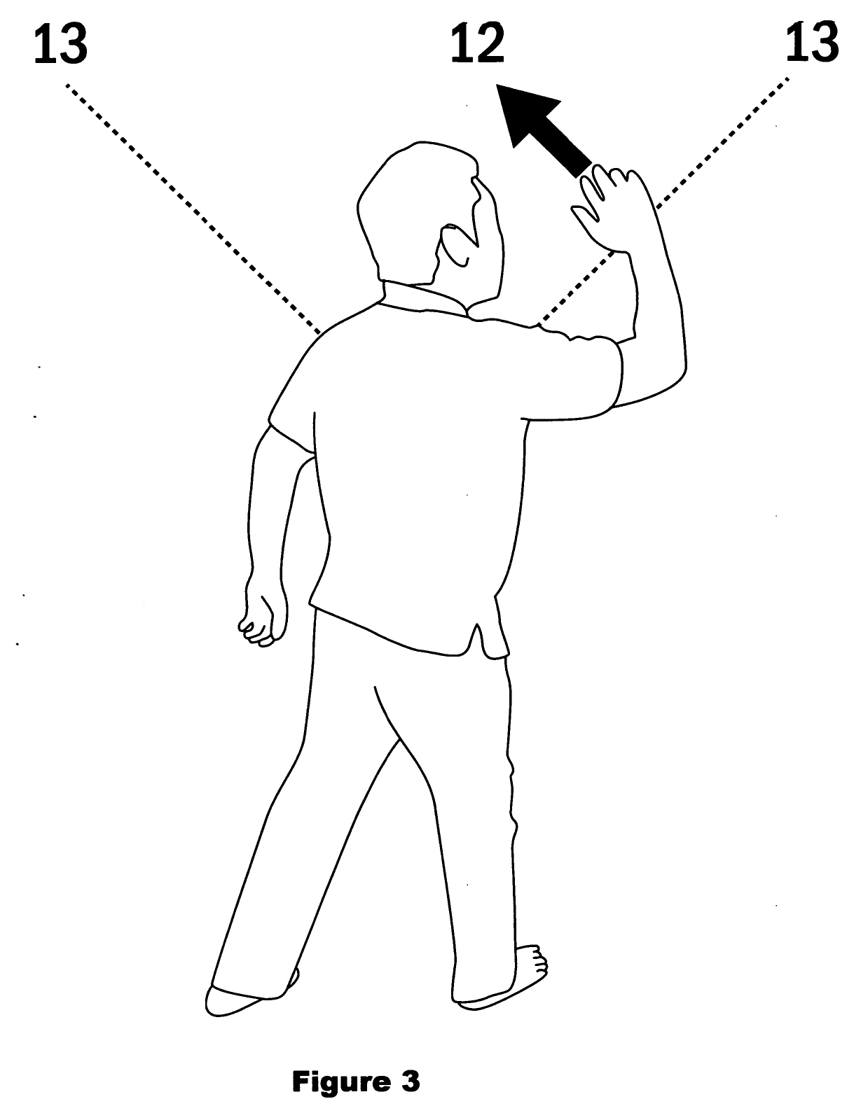

FIG. 3 shows a person standing and the relative position of the hand to the face.

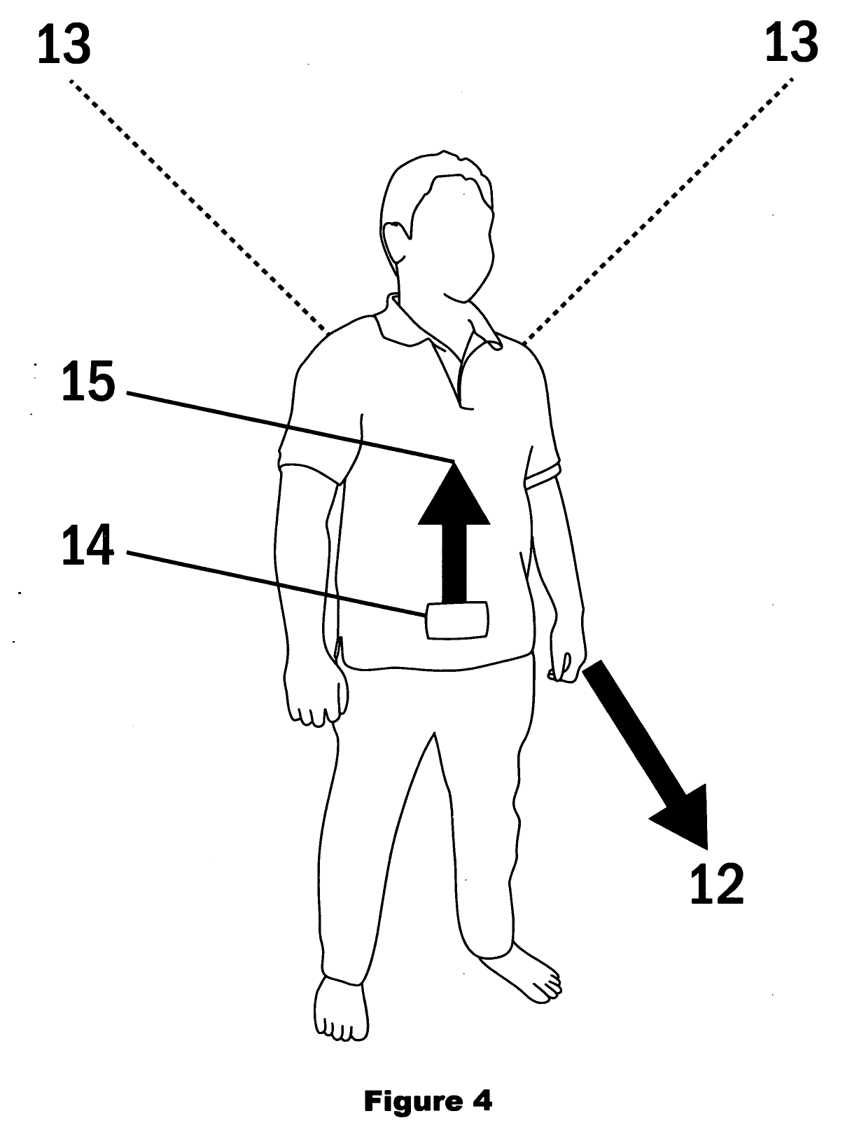

FIG. 4 shows a person standing with a secondary device that measures the position of the body.

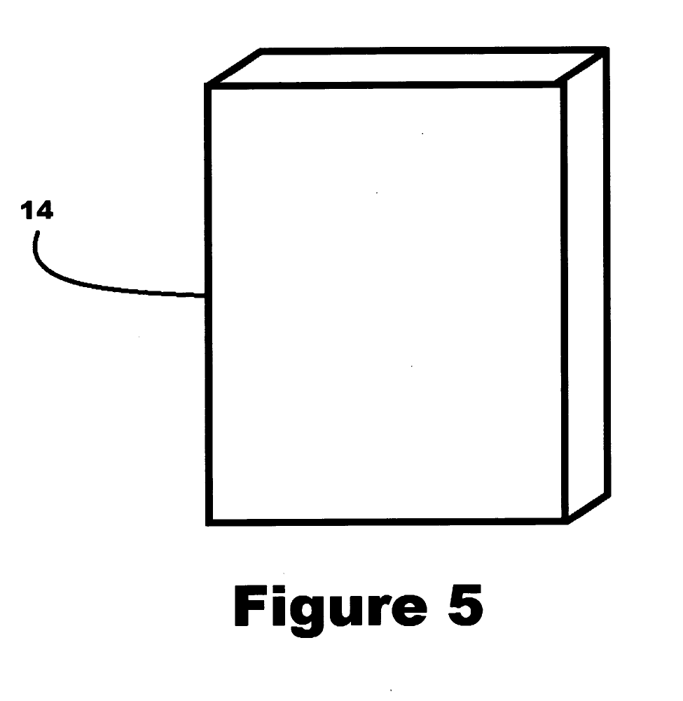

FIG. 5 is a device used to measure the position of the body.

### DRAWINGS

### Reference Numerals

11 Hand Position Measurement Device
12 Position of Hand
13 Position Too Close to Face
14 Secondary Measurement Device
15 Position of Body

### DETAILED DESCRIPTION

### First Embodiment

FIG. 1 is a device worn by an individual that finds the position of the hand. Reference Number 11 is a device that will find the position of the hand of an individual.

FIG. 2 shows a person standing. Reference Number 12 shows the angle of the hand. Reference Number 13 is an angle for the hand that is too close to the face.

FIG. 3 shows the angle of the hand passing the angle too close to the face.

### OPERATION

### First Embodiment

In one embodiment, a person is assumed to be sitting or standing. In these positions, an angle formed from the face of the person to the ground is close to perpendicular. An angle formed from the hand of the person to the ground is shown in Reference Number 12. An angle from the ground, Reference Number 13, is chosen. If the angle of the hand approaches the angle in Reference Number 13, the hand would approach the face. In this embodiment, the device would warn the individual that the hand is approaching the face.

The method of measuring the angle of the hand or body can include, but is not limited to, an accelerometer, gyroscope, tilt sensor, or mercury switch.

The method of warning the individual can include, but is not limited to, an audible tone, vibration, LED pulsing, or communication with another device.

A controller is preferably used to process information received from the sensors, and may include a microprocessor, and can further employ methods that can decrease the incidence of a false alarm.

In other applications, the sensor assembly can modify or reverse undesirable behavior, such as trichotillomania, nail-biting, etc. Although the sensor assembly as described herein is particularly useful for human beings, it is recognized that it can effectively be utilized with domesticated or non-domesticated animals.

### DESCRIPTION

### Alternative Embodiment

FIG. 4 shows a secondary device worn on the body. FIG. 5 shows a secondary device.

### OPERATION

### Alternative Embodiment

In an alternate embodiment, a secondary device, Reference Number 14, is worn on the body. The secondary device measures the angle of the body in free space, Reference Number 15. The secondary device communicates with the hand position measurement device described above. The relative positions of the body, hand, and face can be calculated from the positions of the devices. The advantage of having the secondary device is that the individual can be leaning, lying down, or any other position. It will also be possible to get more precise angles that are needed by either hand to approach the face. In this embodiment, the device would warn the individual that the hand is approaching the face.

The secondary device can be worn anywhere on the body, including but not limited to, the torso, neck, or head.

### CONCLUSION, RAMIFICATIONS, AND SCOPE

Accordingly, the reader will see that the hand position sensor of the various embodiments can be used to prevent hand-to-face-contact. It can be discretely worn on the wrist and does not require any additional components in one embodiment. In an alternate embodiment, a secondary device can also be placed in a discrete location to provide additional accuracy when needed. The chance of a false positive is reduced since the hand is being tracked.

I claim:

1. A method of preventing hand-to-face contact comprising:
Providing a person at least one sensor which detects the position of his/her hand,
specifying a position of the hand that would be too close to the person's face,
warning said person if his/her hand approaches the position specified,
whereby the step of reducing of hand-to-face contacts by said person reduces the risk of viral related infections or other undesirable effect.
